(19) United States
(12) Patent Application Publication
(10) Pub. No.: US 2021/0096121 A1
(43) Pub. Date: Apr. 1, 2021

(54) METHOD AND DEVICE FOR DETECTING WATER QUALITY BASED ON COLOR RECOGNITION, AND STORAGE MEDIUM

(71) Applicant: Wuyi University, Jiangmen (CN)

(72) Inventors: Jingling Zhang, Jiangmen (CN); Zhanran Nie, Jiangmen (CN); Yingjian Wu, Jiangmen (CN); Tianlei Wang, Jiangmen (CN); Tao Ou, Jiangmen (CN); Lin Shi, Jiangmen (CN); Jintao Huang, Jiangmen (CN)

(21) Appl. No.: 16/944,602

(22) Filed: Jul. 31, 2020

(30) Foreign Application Priority Data
Sep. 26, 2019 (CN) 2019109167883

(51) Int. Cl. G01N 33/18; G01N 21/80; G06T 7/90

(52) U.S. Cl. CPC G01N 33/18; G06T 7/90; G01N 21/80

(57) ABSTRACT

A method and device for detecting water quality based on color recognition, and a storage medium. The method includes: collecting a water quality parameter sample and a RGB color parameter sample; normalizing the water quality parameter sample and the RGB color parameter sample; fitting a non-linear curve formed by RGB-PH values by using a RBF neural network model; establishing a RGB-water quality parameter lightweight database by using the non-linear curve of the RGB-water quality parameter; acquiring a color RGB value after a water sample to be detected reacts with a reagent; and comparing the color RGB value with the database to obtain a water quality detection parameter of the water sample to be detected.

### CROSS-REFERENCE TO RELATED APPLICATIONS

This application is based on and claims the benefit of priority from Chinese Patent Application No. 2019109167883, filed on 26 Sep. 2019, the entirety of which is incorporated by reference herein.

### TECHNICAL FIELD

The disclosure relates to the field of water quality detection technologies, and more particularly, to a method, apparatus, device for detecting water quality based on color recognition, and a storage medium.

### BACKGROUND

At present, a water quality should be regularly detected every day for freshwater aquaculture, and it is generally necessary to detect parameters such as a PH value, a turbidity, nitrite, ammonia nitrogen and dissolved oxygen. The water quality is adjusted according to detection results, so as to ensure normal growth of aquaculture products.

At present, there are two methods mainly used for water quality detection of freshwater aquaculture: one is to use a reagent to detect the water quality manually, and the other is to use an electronic sensor to detect the water quality.

For the first method, the detection results are obtained by manually taking a water sample, dropping the reagent into the water sample, and comparing the water sample with a standard colorimetric card with human eyes. However, since the human eyes are easily affected by other factors, and may feel different about the same color in different environments, there may be an error in color judgment. Moreover, multiple detections are required every day, thus being time-consuming and laborious. In addition, comparison with the colorimetric card is required when the reagent is used to detect the water quality manually. However, the matched colorimetric card only has fixed colors corresponding to fixed water quality parameter values. There is a large span between water quality parameter grades, and in case of spanning across one water quality parameter grade, the water quality parameters are generally unable to be accurately read.

For the second method above, the water quality is detected by the electronic sensor, which is expensive in price, high in detection accuracy, complex in operation and high in maintenance cost. Moreover, a farmer needs a variety of sensors, and an overall cost of a whole set of sensors is too high.

### SUMMARY

The disclosure is intended to solve at least one of the technical problems in the prior art. Therefore, the disclosure provides a method, apparatus and device for detecting water quality based on color recognition and a storage medium, a non-linear curve is able to be fitted through a RBF neural network, and a water quality parameter is measured by establishing a RGB-water quality parameter lightweight database by using the non-linear curve to perform color recognition, so that measurement is more accurate.

A method for detecting water quality based on color recognition is provided in a first aspect of the disclosure including:

collecting a water quality parameter sample and a RGB color parameter sample;

normalizing the water quality parameter sample and the RGB color parameter sample;

fitting a non-linear curve formed by a RGB-water quality parameter by using a RBF neural network model;

establishing a RGB-water quality parameter lightweight database by using the non-linear curve of the RGB-water quality parameter;

acquiring a color RGB value after a water sample to be detected reacts with a reagent; and

comparing the color RGB value with the lightweight database to obtain a water quality detection parameter of the water sample to be detected.

The method for detecting water quality based on color recognition of the disclosure has at least the following beneficial effects: in the embodiment, a color recognition technology is used to replace human eyes to acquire color information with water quality parameter information; reagent detection is used to overcome a defect of high price of a current electronic sensor; the non-linear curve formed by the RGB-water quality parameter is fitted by using the RBF neural network, such as fitting the non-linear curve formed by the RGB-PH values, and limitation of colorimetric card reading is broken through, so that the water quality parameter is accurately measured.

In some embodiments, the method for detecting water quality based on color recognition further includes:

in response to the water quality detection parameter exceeding a preset water quality threshold, sending an alarm message.

In some embodiments, the comparing the color RGB value with the lightweight database includes using a minimum absolute value method for comparison. The RGB-water quality parameter lightweight database is established by using the non-linear curve of the RGB-water quality parameter, and data are compared by using the minimum absolute value method, so as to realize off-line and instant RGB-water quality parameter recognition of a single-chip microcomputer.

A apparatus for detecting water quality based on color recognition is provided in a second aspect of the disclosure including:

a collection unit configured to collect a water quality parameter sample and a RGB color parameter sample;

a normalization unit configured to normalize the water quality parameter sample and the RGB color parameter sample;

a curve fitting unit configured to fit a non-linear curve formed by a RGB-water quality parameter by using a RBF neural network model;

a database establishing unit configured to establish a RGB-water quality parameter lightweight database by using the non-linear curve of the RGB-water quality parameter;

an acquisition unit configured to acquire a color RGB value after a water sample to be detected reacts with a reagent; and

a comparison unit configured to compare the color RGB value with the database to obtain a water quality detection parameter of the water sample to be detected.

In some embodiments, the apparatus for detecting water quality based on color recognition further includes the following unit:

an alarm unit configured to, when the water quality detection parameter exceeds a preset water quality threshold, send an alarm message.

In some embodiments, the comparison unit is further configured to compare the color RGB value with the lightweight database by using a minimum absolute value method.

A device for detecting water quality based on color recognition is provided in a third aspect of the disclosure, including at least one control processor and a memory for communicating with the at least one control processor, wherein the memory stores an instruction executable by the at least one control processor, and the instruction is executed by the at least one control processor, so that the at least one control processor is able to execute the method for detecting water quality based on color recognition of the first aspect above.

A computer-readable storage medium is provided in a fourth aspect of the disclosure, wherein the computer-readable storage medium stores a computer-executable instruction, and the computer-executable instruction is used for enabling a computer to execute the method for detecting water quality based on color recognition of the first aspect above.

A computer program product is provided in a fifth aspect of the disclosure, wherein the computer program product includes a computer program stored on a computer-readable storage medium, the computer program includes a program instruction, and when the program instruction is executed by a computer, the computer executes the method for detecting water quality based on color recognition of the first aspect above.

The additional aspects and advantages of the disclosure will be partially provided in the following description, and will partially be apparent in the following description, or learned by practice of the disclosure.

### BRIEF DESCRIPTION OF THE DRAWINGS

The above and/or additional aspects and advantages of the disclosure will be apparent and easily understood from the description of the embodiments with reference to the following accompanying drawings, wherein:

FIG. 1 is a diagram of a method for detecting water quality based on color recognition according to an embodiment of the disclosure;

FIG. 2 is a diagram of an apparatus for detecting water quality based on color recognition according to an embodiment of the disclosure; and

FIG. 3 is a diagram of a device for detecting water quality based on color recognition according to an embodiment of the disclosure.

### REFERENCE NUMERALS

100 apparatus for detecting water quality based on color recognition
110 collection unit
120 normalization unit
130 curve fitting unit
140 database establishing unit
150 acquisition unit
160 comparison unit
170 alarm unit
200 device for detecting water quality based on color recognition
210 control processor
220 memory

### DETAILED DESCRIPTION

Reference will now be made in detail to the embodiments of the present invention, examples of which are illustrated in the accompanying drawings, wherein like reference numerals refer to the same or similar elements or elements having the same or similar functions throughout. The embodiments described below with reference to the accompanying drawings are illustrative only for the purpose of explaining the disclosure and are not to be construed as limiting the disclosure.

In the description of the disclosure, it should be understood that the positional descriptions referred to, for example, the directional or positional relationships indicated by upper, lower, front, rear, left, right, etc., are based on the directional or positional relationships shown in the drawings, and are only for convenience and simplification of description of the disclosure, but not for indicating or implying that the referred device or element must have a specific direction, be constructed and operated in a specific direction, and thus should not be construed as limiting the disclosure.

In the description of the disclosure, “certain” means one or more, “a plurality of” means two or more, and “greater than”, “less than”, “more than”, etc. are understood as excluding the number itself, “above”, “below”, “within”, etc. are understood as including the number itself. “First”, “second”, etc., if referred to, are for the purpose of distinguishing technical features only, cannot be understood as indicating or implying a relative importance or implicitly indicating the number of technical features indicated or implicitly indicating the precedence of technical features indicated.

In the description of the disclosure, unless otherwise clearly defined, terms such as “arrange”, “mount”, “connect” should be understood in a broad sense, and those skilled in the art can reasonably determine the specific meanings of the above terms in the disclosure by combining the specific contents of the technical solutions.

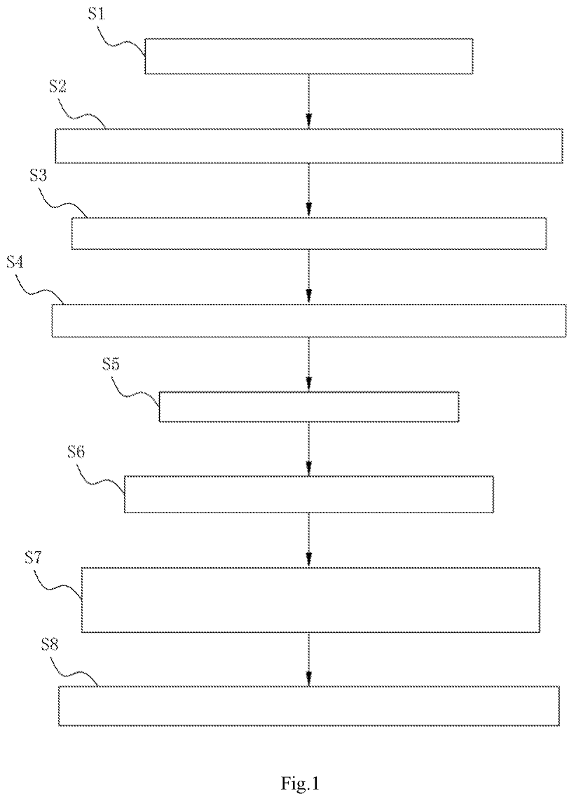

With reference to FIG. 1, a method for detecting water quality based on color recognition is provided in a first aspect of the disclosure which includes the following steps.

At step S1, a water quality parameter sample and a RGB color parameter sample are collected. Water quality parameters to be detected include a PH value, a turbidity, nitrite, ammonia nitrogen, dissolved oxygen and the like. Taking the PH value as an example, a traditional acid-base neutralization titration method is used to prepare reagent solutions with different PH values, and the reagent solutions are put into a water quality detection apparatus. The PH value ranges from 0 to 14, and the PH value is divided into 28 grades at an interval of 0.5. The prepared PH reagent solutions are put into the water quality detection apparatus to obtain corresponding RGB color parameters by using the water quality detection apparatus. In order to reduce errors and contingencies in an experiment, 10 groups are recorded with the same operation, and 280 RGB-PH value samples are obtained.

At step S2, the water quality parameter sample and the RGB color parameter sample are normalized. For a value of the RGB color parameter sample, a standardized color value between 0 to 1 is obtained by dividing by 255. For a value of a PH sample of the water quality parameter, a standardized color value between 0 to 1 is obtained by dividing by 14.

At step S3, a non-linear curve formed by a RGB-water quality parameter is fitted by using a RBF neural network model. Three input nodes are selected, which respectively correspond to three channels of the RGB color parameter. Twelve hidden nodes are selected in a middle hidden layer. One output node is provided, which corresponds to the PH value of the water quality parameter. The RBF neural network is utilized, gradient descent is performed on a loss function, and each parameter is constantly revised by a trial and error method, so as to finally obtain a corresponding relationship between the red, green and blue channels of the RGB color parameter and the PH value of the water quality parameter, and a RGB-PH value neural model, and the non-linear curve formed by the RGB-PH values is fitted. Moreover, the non-linear curves of different RGB-water quality parameters are respectively fitted by the same method.

At step S4, a RGB-water quality parameter lightweight database is established by using the non-linear curve of the RGB-water quality parameter. The non-linear curve is discretized according to a PH value accuracy of 0.01 to obtain a data table with a set of RGB corresponding to one PH value, and the RGB-PH value lightweight database is established. Moreover, different RGB-water quality parameter lightweight databases are obtained by the same method respectively.

At step S5, the lightweight database is written into a Flash of a single chip microcomputer, which is convenient for off-line and instant RGB-water quality parameter recognition.

At step S6, a color RGB value is acquired after a water sample to be detected reacts with a reagent.

At step S7, the color RGB value is compared with the lightweight database by using a minimum absolute value method to obtain a water quality detection parameter of the water sample to be detected.

The method for detecting water quality based on color recognition according to the embodiment of the disclosure has at least the following beneficial effects: in the embodiment, a color recognition technology is used to replace human eyes to acquire color information with water quality parameter information. Reagent detection is used to overcome a defect of high price of a current electronic sensor. The non-linear curve formed by the RGB-water quality parameter is fitted by using the RBF neural network, such as fitting the non-linear curve formed by the RGB-PH values, and limitation of colorimetric card reading is broken through, so that the water quality parameter is accurately measured. The RGB-water quality parameter lightweight database is established by using the non-linear curve of the RGB-water quality parameter, and data are compared by using the minimum absolute value method, so as to realize off-line and instant RGB-water quality parameter recognition of a single-chip microcomputer.

In some embodiments according to the first aspect of the disclosure, the method for detecting water quality based on color recognition further includes the following step.

At step S8, when the water quality detection parameter exceeds a preset water quality threshold, an alarm message is sent.

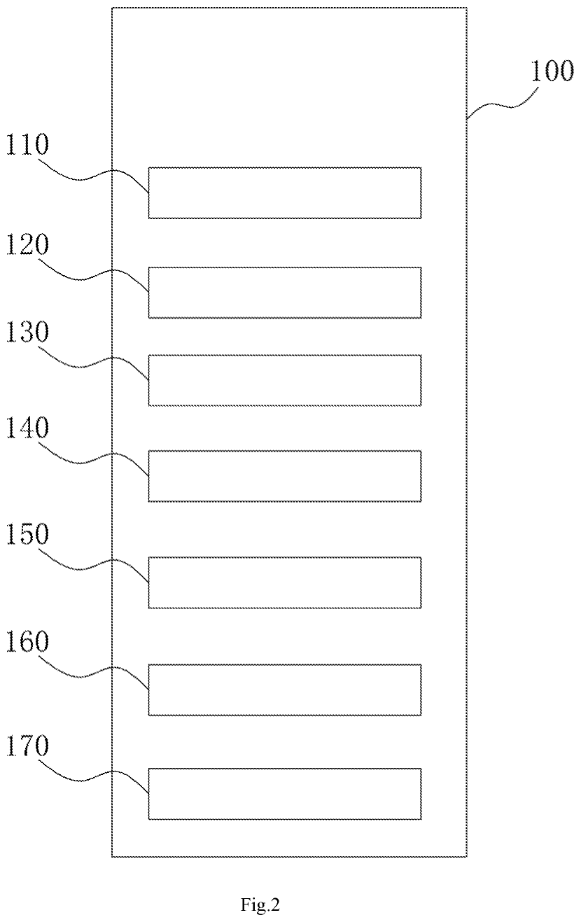

With reference to FIG. 2, an apparatus for detecting water quality based on color recognition 100 is provided in a second aspect of the disclosure including:

a collection unit 110 configured to collect a water quality parameter sample and a RGB color parameter sample;

a normalization unit 120 configured to normalize the water quality parameter sample and the RGB color parameter sample;

a curve fitting unit 130 configured to fit a non-linear curve formed by a RGB-water quality parameter by using a RBF neural network model;

a database establishing unit 140 configured to establish a RGB-water quality parameter lightweight database by using the non-linear curve of the RGB-water quality parameter;

an acquisition unit 150 configured to acquire a color RGB value after a water sample to be detected reacts with a reagent;

a comparison unit 160 configured to compare the color RGB value with the lightweight database to obtain a water quality detection parameter of the water sample to be detected.

It should be noted that since the apparatus for detecting water quality based on color recognition 100 in this embodiment is based on the same inventive concept as the above method for detecting water quality based on color recognition, the corresponding contents in the method embodiment are also applicable to the apparatus embodiment, which will not be described in detail herein.

In some embodiments according to the second aspect of the disclosure, the apparatus for detecting water quality based on color recognition further includes:

an alarm unit 170 configured to, when the water quality detection parameter exceeds a preset water quality threshold, send an alarm message.

In some embodiments according to the second aspect of the disclosure, the comparison unit 160 is further configured to compare the color RGB value with the lightweight database by using a minimum absolute value method.

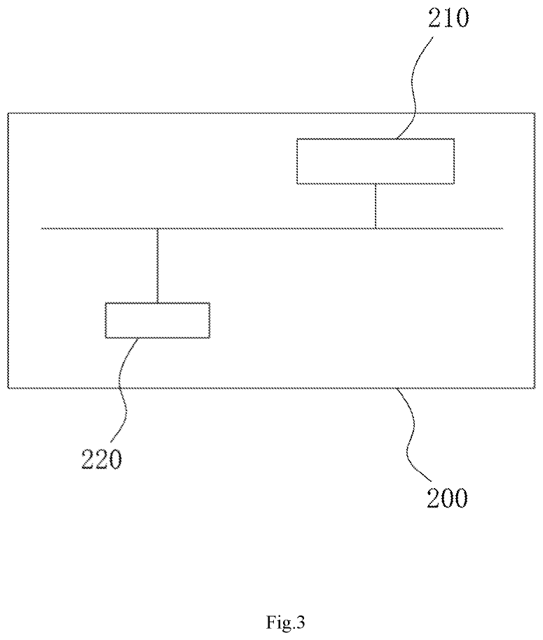

With reference to FIG. 3, a device for detecting water quality based on color recognition 200 is provided in a third aspect of the disclosure, and the device for detecting water quality based on color recognition 200 may be any type of intelligent terminal, such as a mobile phone, a tablet computer, a personal computer, and the like.

Specifically, the device for detecting water quality based on color recognition 200 includes: one or more control processors 210 and a memory 220. One control processor 210 is taken as an example in FIG. 3.

The control processor 210 and the memory 220 may be connected by a bus or in other manners. Connection by the bus is taken as an example in FIG. 3.

The memory 220, as a non-transient computer-readable storage medium, may be used for storing a non-transient software program, a non-transient computer-executable program and a module, such as a program instruction/module corresponding to the method for detecting water quality based on color recognition in the embodiment of the disclosure, such as units 110 to 170 shown in FIG. 2. The control processor 210 executes various functional applications and data processing of the apparatus for detecting water quality based on color recognition 100 by operating the non-transient software program, instruction and module stored in the memory 220, which means that, the method for detecting water quality based on color recognition in the above method embodiment is realized.

The memory 220 may include a program storing area and a data storing area, wherein the program storing area may store an application program required by an operating system and at least one function, and the data storing area may store data created for use according to the apparatus for detecting water quality based on color recognition 100, and the like. In addition, the memory 220 may include a high-speed random access memory, and may also include a non-transient memory, such as at least one disk memory device, flash memory device, or other non-transient solid-state memory devices. In some embodiments, the memory 220 may optionally include a memory remotely arranged relative to the control processor 210, and these remote memories may be connected to the device for detecting water quality based on color recognition 200 through a network. Examples of the above network include but are not limited to the Internet, the intranet, the local area network, the mobile communication network and a combination thereof.

The one or more modules are stored in the memory 220 which, when executed by the one or more control processors 210, cause to execute the method for detecting water quality based on color recognition in the above method embodiment, such as execute the method steps S1 to S8 in FIG. 1 in the above description, so as to realize functions of the units 110 to 170 in FIG. 2.

A computer-readable storage medium is provided in a fourth aspect of the disclosure. The computer-readable storage medium stores a computer-executable instruction, and the computer-executable instruction is executed by one or more control processors 210. For example, when the computer-executable instruction is executed by one control processor 210 in FIG. 3, the above one or more control processors 210 may execute the method for detecting water quality based on color recognition in the above method embodiment, such as execute the method steps S1 to S8 in FIG. 1 in the above description, so as to realize functions of the units 110 to 170 in FIG. 2.

The apparatus embodiment described above is only exemplary, wherein the units described as separate components may or may not be physically separated, which means that the units may be located in one place or distributed to multiple network units. Some or all of the modules may be selected according to actual needs to achieve the objectives of the solutions in the embodiments.

From the description of the above embodiments, those skilled in the art may clearly understand that each embodiment may be realized by means of software with a general hardware platform. Those skilled in the art may understand that all or partial flows in the method of the embodiment above may be completed by instructing related hardware through a computer program, and the program may be stored in a computer-readable storage medium. The program may include the flows of the above method embodiment when executed. The storage medium may be a magnetic disk, an optical disk, a Read Only Memory (ROM), a Random Access Memory (RAM), or the like.

A computer program product is provided in a fifth aspect of the disclosure, wherein the computer program product includes a computer program stored on a computer-readable storage medium, and the computer program includes a program instruction. When the program instruction is executed by a computer, the computer executes the method for detecting water quality based on color recognition of the first aspect above.

The embodiments of the disclosure are described in detail with reference to the accompanying drawings above, but the disclosure is not limited to the above embodiments, and various changes may also be made within the knowledge scope of those of ordinary skills in the art without departing from the purpose of the disclosure.

We claim:

1. A method for detecting water quality based on color recognition, comprising:
collecting a water quality parameter sample and a RGB color parameter sample;
normalizing the water quality parameter sample and the RGB color parameter sample;
fitting a non-linear curve formed by a RGB-water quality parameter by using a RBF neural network model;
establishing a RGB-water quality parameter lightweight database by using the non-linear curve of the RGB-water quality parameter;
acquiring a color RGB value after a water sample to be detected reacts with a reagent; and
comparing the color RGB value with the lightweight database to obtain a water quality detection parameter of the water sample to be detected.

2. The method for detecting water quality based on color recognition of claim 1, further comprising:
in response to the water quality detection parameter exceeding a preset water quality threshold, sending an alarm message.

3. The method for detecting water quality based on color recognition of claim 1, wherein the comparing the color RGB value with the lightweight database comprises using a minimum absolute value method for comparison.

4. A device for detecting water quality based on color recognition, comprising at least one control processor and a memory for communicating with the at least one control processor, wherein the memory stores an instruction executable by the at least one control processor, and the instruction is executed by the at least one control processor, so that the at least one control processor is able to execute a method for detecting water quality based on color recognition comprising:
collecting a water quality parameter sample and a RGB color parameter sample;
normalizing the water quality parameter sample and the RGB color parameter sample;
fitting a non-linear curve formed by a RGB-water quality parameter by using a RBF neural network model;
establishing a RGB-water quality parameter lightweight database by using the non-linear curve of the RGB-water quality parameter;
acquiring a color RGB value after a water sample to be detected reacts with a reagent; and
comparing the color RGB value with the lightweight database to obtain a water quality detection parameter of the water sample to be detected.

5. The device for detecting water quality based on color recognition of claim 4, wherein the method further comprises:
in response to the water quality detection parameter exceeding a preset water quality threshold, sending an alarm message.

6. The device for detecting water quality based on color recognition of claim 4, wherein the comparing the color RGB value with the lightweight database comprises using a minimum absolute value method for comparison.

7. A computer-readable storage medium, wherein the computer-readable storage medium stores a computer-executable instruction, and the computer-executable instruction is used for enabling a computer to execute a method for detecting water quality based on color recognition comprising:
collecting a water quality parameter sample and a RGB color parameter sample;
normalizing the water quality parameter sample and the RGB color parameter sample;
fitting a non-linear curve formed by a RGB-water quality parameter by using a RBF neural network model;
establishing a RGB-water quality parameter lightweight database by using the non-linear curve of the RGB-water quality parameter;
acquiring a color RGB value after a water sample to be detected reacts with a reagent; and
comparing the color RGB value with the lightweight database to obtain a water quality detection parameter of the water sample to be detected.

8. The computer-readable storage medium of claim 7, wherein the method further comprises:
in response to the water quality detection parameter exceeding a preset water quality threshold, sending an alarm message.

9. The computer-readable storage medium of claim 7, wherein the comparing the color RGB value with the lightweight database comprises using a minimum absolute value method for comparison.
